(19) United States
(12) Patent Application Publication
(10) Pub. No.: US 2026/0232720 A1
(43) Pub. Date: Aug. 13, 2026

(54) NEW MEDICAL USE OF SEVUPARIN IN THE TREATMENT OF ENDOTOXEMIA

(71) Applicant: Modus Therapeutics AB, Stockholm (SE)

(72) Inventor: John Öhd, Nacka (SE)

(21) Appl. No.: 19/157,218

(22) PCT Filed: Feb. 16, 2024
(86) PCT No.: PCT/EP2024/054006

§ 371 Date: Aug. 15, 2025

(30) Foreign Application Priority Data
Feb. 17, 2023 (GB) 2302278.3
Sep. 15, 2023 (GB) 2314100.5

(51) Int. Cl. A61K 31/727; A61P 11/00; A61P 29/00; A61P 31/04

(52) U.S. Cl. CPC A61K 31/727; A61P 11/00; A61P 29/00; A61P 31/04

(57) ABSTRACT

The present invention is directed to the compound sevuparin or a pharmaceutically acceptable salt thereof, for use in the treatment of endotoxemia, as well as to one or more medical conditions caused by or associated with endotoxemia, such as endotoxemia-associated Acute Respiratory Distress Syndrome (ARDS) and endotoxemia-associated Acute Lung Injury (ALI).

### FIELD OF THE INVENTION

The present invention is directed to the compound sevuparin or a pharmaceutically acceptable salt thereof, for use in the treatment of endotoxemia as well as to one or more medical conditions caused by or associated with endotoxemia, such as endotoxemia-associated Acute Respiratory Distress Syndrome (ARDS) and endotoxemia-associated Acute Lung Injury (ALI).

### BACKGROUND OF THE INVENTION

Elevated levels of endotoxin in the blood are present in some, but not all patients with sepsis, as well as in many patients with acute life-threatening illnesses that would not meet the criteria for sepsis. A majority of patients meeting clinical criteria for sepsis can be shown to have elevated levels of circulating endotoxin; however, these elevated levels are also seen in a large number of acutely ill patients who do not meet sepsis criteria.

Circulating endotoxin appears to be present in most patients who meet classical clinical criteria for sepsis (Brock-Utne J G et al: S Afr Med J 1988; 73: pp. 533-536; Hasday J D et al: Chest 1999; 115: pp. 829-835) although several authors have suggested otherwise (Opal S M et al: Crit Care Med 2003; 31 (Suppl 1): pp. 57-64; Cohen J: Intensive Care Med 2000; 26 (Suppl 1):S51-S56).

Endotoxin, or more accurately termed bacterial lipopolysaccharide (LPS), is recognized as the most potent microbial mediator implicated in the pathogenesis of sepsis and septic shock. Yet despite its discovery well over a century ago, the fundamental role of circulating endotoxin in the blood of most patients with septic shock remains enigmatic and a subject of considerable controversy. LPS is the most prominent ‘alarm molecule’ sensed by the host's early warning system of innate immunity presaging the threat of invasion of the internal milieu by Gram-negative bacterial pathogens. In small doses within a localized tissue space, LPS signaling is advantageous to the host in orchestrating an appropriate antimicrobial defense and bacterial clearance mechanisms. Conversely, the sudden release of large quantities of LPS into the bloodstream is clearly deleterious to the host, initiating the release of a dysregulated and potentially lethal array of inflammatory mediators and procoagulant factors in the systemic circulation. The massive host response to this single bacterial pattern recognition molecule is sufficient to generate diffuse endothelial injury, tissue hypoperfusion, disseminated intravascular coagulation and refractory shock (Ronco C et al; Endotoxemia and Endotoxin Shock: Disease, Diagnosis and Therapy. Contrib Nephrol. Basel, Karger, 2010, vol 167, pp 14-24). The most potent of all the pathogen-associated molecular pattern (PAMP) molecules is bacterial lipopolysaccharide (LPS), also known as endotoxin.

Endotoxin concentrations can vary widely in individuals depending on health status, presence of stresses such as intestinal hypoxia or hemorrhagic shock, acute conditions such as pancreatitis, chronic conditions such as periodontitis, presence of infections, liver competency, etc. Otherwise healthy individuals have been observed with transient mild endotoxemia (increased endotoxin concentrations of 5-15 pg/ml) after physical stress such as athletic competition of long duration (Camus, G., et al. (1997) Clin. Sci (Lond.) 92, 415-422; Jeukendrup, A. E., et al. (2000) Clin. Sci Lond. 98, 47-55). Hung et al (Acta Microbiologica et Immunologica Hungarica, 49 (1), pp. 151-157 (2002)) determined the plasma level of endotoxin in 116 healthy blood donors, and this was less than 1 EU/ml (in the range of 0.01-1.0 EU/ml) in most of these healthy donors, but was always measurable. The wording EU stands for Endotoxin Units.

Endotoxin levels have been observed to vary widely in some critically ill patients. ICU patients fulfilling criteria for severe sepsis or septic shock have been observed with higher endotoxin levels of 310+/−810 pg/ml and 470+/−57 pg/ml. However, critically ill patients not diagnosed with sepsis also showed elevated endotoxin levels of 157+/−140 pg/ml, but no patients with gram negative infection exhibited an endotoxin level below 50 pg/ml (Venet, C., et al. (2000) Intensive Care Med. 26, pp. 538-544: Marshall, J. C., et al. (2002) Crit. Care 6, pp. 289-290). Endotoxin levels in patients suffering from systemic meningococcal disease reached levels greater than 700 pg/ml, and were associated with increased development of septic shock, adult respiratory distress and death. Thus, endotoxin concentrations are elevated to varying degrees in patients suffering from a variety of diseases and conditions.

The presence of endotoxin in the circulation leads to altered cardiovascular function, tachypnoea and lung dysfunction as well as acute kidney injury. These symptoms are also overlapping in other critical conditions such as sepsis and septic shock without notable ongoing endotoxemia. However, it is believed that when endotoxemia occurs, or is present, on top of other critical conditions, it contributes significantly to the worsening of an endotoxemia related condition.

Research has consistently showed that subjects with endotoxemia rapidly develops an increased respiratory rate lasting for up to 24 h following systemic exposure. Chen J S et al discloses that NSAIDs, such as ibuprofen, which act on cyclooxygenases, can dampen the elevated respiratory rate usually seen in a subject injected with LPS (Journal of Virology April 2021; Vol 95; Issue 7; pp. 1-16; e00014-21). It has been suggested that the rapid onset respiratory symptoms upon endotoxemia is due to the pulmonary vasculature endothelium being specifically sensitive to damage by endotoxin as shown in rodent models where the time kinetic for signs of endothelial damage is in line with that of observed respiratory symptoms in man as described above (Schmidt et al, Nat Med. 2012 August; 18 (8): 1217-23. doi: 10.1038/nm.2843. Epub 2012 Jul. 22). The development of endotoxin induced pulmonary endothelial damage is intimately connected with detrimental effects on immune mechanisms as the damage causes sequestration and relative depletion of white blood cells (neutrophils) in the circulation which in turn risks to hamper the capacity to resolve severe inflammatory states such as endotoxemia and sepsis (Schmidt et al, Nat Med. 2012 August; 18 (8): 1217-23. doi: 10.1038/nm.2843. Epub 2012 Jul. 22). Indeed, endotoxin induced depletion of other immune cells such as basophils and lymphocytes may also be associated with an impaired ability to resolve severe endotoxemia related states as basophil levels are directly linked to outcomes in both rodents and man (Piliponsky et al, Nat Immunol. 2019 February; 20 (2): pp. 129-140) and lymphopenia similarly predicts immunosuppression and poor outcome in patients (de Jager et al., Crit Care, 2010; 14 (5), Drewry et al, Shock. 2014 November; 42 (5): pp. 383-391).

In Langenbecks Arch Surg 2008, 393: pp. 473-478, Buttenschoen et al, disclose that exaggerated cytokine release of mononuclear cells has been observed in acute lung injury/acute respiratory distress syndrome (ALI/ARDS).

In FASEB Journal, Vol. 33 September 2019, pp. 10443-10452, Rasmuson J et al suggest that the use of low-anticoagulant heparin derivatives such as sevuparin, could be a promising strategy to reduce tissue damage and respiratory distress caused by neutrophils without hampering the recruitment of the phagocytic cells for antimicrobial actions.

### BRIEF DESCRIPTION OF THE INVENTION

An aspect of the present invention is to provide a novel therapy for endotoxemia. More particularly the invention is directed to the compound sevuparin or a pharmaceutically acceptable salt thereof, for use in the treatment of endotoxemia.

Yet an aspect of the invention is to provide a novel therapy for the treatment of a medical condition comprising elevated levels of endotoxin in a subject.

Yet an aspect of the invention is the compound sevuparin or a pharmaceutically acceptable salt thereof, for use in the prophylactic treatment in a subject at risk of developing endotoxemia as well as a medical condition caused by or associated with endotoxemia.

### BRIEF DESCRIPTION OF DRAWINGS

FIG. 1 is a graph showing the lymphocyte count from repeated blood samplings over time in subjects having received LPS intravenously, and simultaneously treatment by a continuous intravenous infusion of either placebo or three different doses of intradermal sevuparin.

FIG. 2 is a graph showing the respiratory rate over time in subjects having received either of placebo or one of three different doses of sevuparin intravenously.

### DETAILED DESCRIPTION OF THE INVENTION

An aspect of the present invention is a chemically modified heparin having:
(i) an antifactor IIa activity of up to 10 IU/mg;
(ii) an antifactor Xa activity of up to 10 IU/mg;
(iii) a weight average molecular weight from 6.5 to 9.5 kDa;
(iv) wherein the polysaccharide chains in said chemically modified heparin contain a predominant structure of the formula:

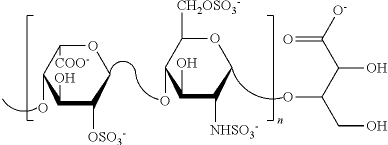

wherein n is an integer of from 2 to 25, such that it comprises from 2 to 25 disaccharide units corresponding to molecular weights from 1.2 to 15 kDa;
(v) retain at least 90% of the sulfate groups compared to native heparin;
(vi) have a reduction in chemically intact pentasaccharide sequences, responsible for providing an antithrombin mediated anticoagulant effect, compared to the polysaccharide chains of native heparin; and
(vii) have a reduction in unsulfated iduronic acid units and glucuronic acid units compared to native heparin;
   for use in the treatment of endotoxemia.

In a further aspect, the chemically modified heparin as used in therapy of endotoxemia as herein described and claimed, has in a 1H-NMR spectrum, no unidentified signals in the ranges 0.10-2.00 ppm, 2.10-3.10 ppm and 5.70-8.00 ppm larger than 4 percent when compared to the height of the signal present in native heparin at 5.42 ppm.

In yet an aspect of the present invention, the predominantly occurring polysaccharide chains in the chemically modified heparin as used in accordance with the invention, have from 6 to 16 disaccharide units with molecular weights from 3.6 to 9.6 kDa.

In yet an aspect of the invention, the chemically modified heparin as used in accordance with the invention, comprises glycol-split residues of the chemical structure:

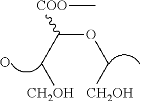

In yet an aspect of the invention, in the chemically modified heparin as used in accordance with the invention, at least 30% of the polysaccharide chains have a molecular weight of at least 8 kDa.

In yet an aspect of the invention, in the chemically modified heparin as used in accordance with the invention, 3-15% of the polysaccharide chains have a molecular weight of at least 15 kDa.

In yet an aspect of the invention, in the chemically modified heparin as used in accordance with the invention, 25-47% of the polysaccharide chains have a molecular weight of at least 9 kDa.

In yet an aspect of the invention, in the chemically modified heparin as used in accordance with the invention, 40-60% of the polysaccharide chains have a molecular weight of at least 7 kDa.

In yet an aspect of the invention, in the chemically modified heparin as used in accordance with the invention, 60-80% of the polysaccharide chains have a molecular weight of at least 5 kDa.

In yet an aspect of the invention, in the chemically modified heparin as used in accordance with the invention, at least 85% of the polysaccharide chains have a molecular weight of at least 3 kDa. In yet an aspect of the invention, in the chemically modified heparin as used in accordance with the invention, at least 95% of the polysaccharide chains have a molecular weight of at least 2 kDa.

An aspect of the present invention is the compound sevuparin for use as therapeutic treatment of endotoxemia.

Yet an aspect of the present invention is the compound sevuparin for use as prophylactic treatment in a subject at risk of developing endotoxemia.

An aspect of the invention is the compound sevuparin for use in the treatment of endotoxemia, wherein the endotoxemia occurs in conjunction with an infection by gram-negative bacteria.

An aspect of the invention is the compound sevuparin for use in the treatment of endotoxemia, wherein the endotoxemia occurs in conjunction with an infection by gram-positive bacteria.

An aspect of the invention is the compound sevuparin for use in the treatment of endotoxemia, wherein the endotoxemia occurs in conjunction with sepsis.

An aspect of the invention is the compound sevuparin for use in the treatment of endotoxemia, wherein the endotoxemia occurs in conjunction with septic shock.

An aspect of the invention is the compound sevuparin for use in the treatment of endotoxemia, wherein the endotoxemia occurs in conjunction with a major surgical procedure such as a thoracic (cardiac) surgery, an abdominal surgery or a neurosurgical procedure.

An aspect of the invention is the compound sevuparin for use in the treatment of endotoxemia, wherein the endotoxemia occurs in conjunction with a severe acute disease such as pancreatitis, cholecystitis, intestinal arterial obstruction, portal vein thrombosis, aortic aneurysm rupture, or aortic dissection.

An aspect of the invention is the compound sevuparin for use in the treatment of endotoxemia, wherein the endotoxemia occurs in conjunction with an injury caused by a major trauma.

An aspect of the invention is the compound sevuparin for use in the treatment of endotoxemia, wherein the endotoxemia occurs in conjunction with hepatic failure.

An aspect of the invention is the compound sevuparin for use in the treatment of endotoxemia, wherein the endotoxemia occurs in conjunction with hepatic failure in a non-cirrhotic subject.

An aspect of the invention is the compound sevuparin for use in the treatment of endotoxemia, wherein the endotoxemia occurs in conjunction with hepatic failure in a cirrhotic subject.

An aspect of the invention is the compound sevuparin for use in the treatment of endotoxemia, wherein the endotoxemia occurs in conjunction with hepatorenal syndrome.

An aspect of the invention is the compound sevuparin for use in the treatment of endotoxemia, wherein the endotoxemia occurs in conjunction with intestinal endotoxemia (leaky gut).

An aspect of the invention is the compound sevuparin for use in the treatment of metabolic endotoxemia.

An aspect of the invention is the compound sevuparin for use in the treatment of diet-induced endotoxemia.

An aspect of the invention is the compound sevuparin for use in the treatment of portal endotoxemia.

An aspect of the invention is the compound sevuparin for use in the treatment of endotoxemia, wherein the endotoxemia is present in a subject having an autoimmune disease, such as inflammatory bowel disease (IBD), Crohn's Disease or ulcerative colitis.

An aspect of the invention is the compound sevuparin for use in the treatment of endotoxemia, wherein the endotoxemia is present in a subject having systemic inflammatory response syndrome (SIRS).

An aspect of the invention is the compound sevuparin for use in the treatment of endotoxemia, wherein the endotoxemia is present in a subject having Multiple organ dysfunction syndrome (MODS).

An aspect of the invention is the compound sevuparin for use in the treatment of endotoxemia, wherein the endotoxemia is present in a subject with Covid-19.

An aspect of the invention is the compound sevuparin or a pharmaceutically acceptable salt thereof, for use in the prophylactic or therapeutic treatment in a subject at risk of developing endotoxemia.

Yet an aspect of the invention is the compound sevuparin or a pharmaceutically acceptable salt thereof, for use in the prophylactic or therapeutic treatment of a medical condition associated with endotoxemia.

An aspect of the invention is the compound sevuparin for use in the prophylactic treatment of a subject at risk of developing endotoxemia-associated Acute Respiratory Distress Syndrome (ARDS).

Yet an aspect of the invention is the compound sevuparin for use in the prophylactic treatment of a subject at risk of developing endotoxemia-associated Acute Lung Injury (ALI).

One aspect of the invention is the compound sevuparin for use in the treatment of a subject suffering from endotoxemia-associated Acute Respiratory Distress Syndrome (ARDS).

Yet an aspect of the invention is the compound sevuparin for use in the treatment of a subject suffering from endotoxemia-associated Acute Lung Injury (ALI).

One aspect the invention is the compound sevuparin or a pharmaceutically acceptable salt thereof, for use in the treatment of Acute Respiratory Distress Syndrome (ARDS) in a subject having endotoxemia.

One aspect the invention is the compound sevuparin or a pharmaceutically acceptable salt thereof, for use in the treatment of Acute Lung Injury (ALI) in a subject having endotoxemia.

One aspect of the invention is the compound sevuparin or a pharmaceutically acceptable salt thereof, for use in the treatment of pneumonia in a subject suffering from endotoxemia-associated Acute Respiratory Distress Syndrome (ARDS), which subject may or may not also have sepsis.

An aspect of the invention is the compound sevuparin for use in the treatment of endotoxemia, wherein said use is combination treatment with standard of care (SOC) therapy for endotoxemia.

Examples of standard of care (SOC) therapy for endotoxemia may be selected from an anti-coagulant agent such as a heparin including but not limited to enoxaparin, an antibiotic; an additional endotoxin neutralizing agent such as a TLR-4 receptor antagonist; a cytokine inhibitor such as IL-6, an IL-1, or a TNF; an anti-inflammatory agent such as a non-steroidal agent (NSAID) including but not limited to salicylic acid or a COX-2 inhibitor; an anti-inflammatory agent such as a steroid including but not limited to prednisone, prednisolone, methylprednisolone, triamcinolone or dexamethasone. Standard of care (SOC) therapy for endotoxemia may also be a combination of one or more of the SOC agents listed herein.

One aspect of the invention is the use of the compound sevuparin or a pharmaceutically acceptable salt thereof, for the manufacture of a medicament for the treatment of endotoxemia.

One aspect of the invention is a method for the treatment of endotoxemia, comprising administering a therapeutically effective amount of the compound sevuparin or a pharmaceutically acceptable salt thereof, to a subject in need of such treatment.

One aspect of the invention is a method for the prophylactic treatment of a subject at risk of developing endotoxemia-associated Acute Respiratory Distress Syndrome (ARDS), comprising administering a therapeutically effective amount of the compound sevuparin or a pharmaceutically acceptable salt thereof, to a subject in need of such prophylactic treatment.

One aspect of the invention is a method for the therapeutic treatment of a subject having endotoxemia-associated Acute Respiratory Distress Syndrome (ARDS), comprising administering a therapeutically effective amount of the compound sevuparin or a pharmaceutically acceptable salt thereof, to a subject in need of such therapeutic treatment.

One aspect of the invention is a method for the prophylactic treatment of a subject at risk of developing endotoxemia-associated Acute Lung Injury (ALI), comprising administering a therapeutically effective amount of the compound sevuparin or a pharmaceutically acceptable salt thereof, to a subject in need of such prophylactic treatment.

One aspect of the invention is a method for the therapeutic treatment of a subject having endotoxemia-associated Acute Lung Injury (ALI), comprising administering a therapeutically effective amount of the compound sevuparin or a pharmaceutically acceptable salt thereof, to a subject in need of such therapeutic treatment.

A further aspect of the invention is a method for the prophylactic treatment of Acute Respiratory Distress Syndrome (ARDS) in a subject at risk of developing endotoxemia-associated Acute Respiratory Distress Syndrome (ARDS), comprising administering a therapeutically effective amount of the compound sevuparin or a pharmaceutically acceptable salt thereof, to a subject in need of such prophylactic treatment.

A further aspect of the invention is a method for the therapeutic treatment of Acute Respiratory Distress Syndrome (ARDS) in a subject having endotoxemia, comprising administering a therapeutically effective amount of the compound sevuparin or a pharmaceutically acceptable salt thereof, to a subject in need of such therapeutic treatment.

One aspect the invention is a method for the prophylactic treatment of Acute Lung Injury (ALI) in a subject having endotoxemia, comprising administering a therapeutically effective amount of the compound sevuparin or a pharmaceutically acceptable salt thereof, to a subject in need of such prophylactic treatment.

One aspect the invention is a method for the therapeutic treatment of Acute Lung Injury (ALI) in a subject having endotoxemia, comprising administering a therapeutically effective amount of the compound sevuparin or a pharmaceutically acceptable salt thereof, to a subject in need of such therapeutic treatment.

One aspect of the invention is a method for the therapeutic treatment of pneumonia in a subject suffering from endotoxemia-associated Acute Respiratory Distress Syndrome (ARDS), which subject may or may not also have sepsis, comprising administering a therapeutically effective amount of the compound sevuparin or a pharmaceutically acceptable salt thereof, to a subject in need of such therapeutic treatment.

One aspect of the invention is a method for the prophylactic treatment of pneumonia in a subject suffering from endotoxemia-associated Acute Respiratory Distress Syndrome (ARDS), which subject may or may not also have sepsis, comprising administering a therapeutically effective amount of the compound sevuparin or a pharmaceutically acceptable salt thereof, to a subject in need of such prophylactic treatment.

### Definitions

Sevuparin is a heparin derivative with the company compound code DF02. The INN (International Nonproprietary Name) for DF02 is sevuparin sodium. The CAS registry number (RN) for sevuparin is RN 9041-08-1.

More specifically, sevuparin is a chemically modified heparin having:
(i) an antifactor IIa activity of up to 10 IU/mg;
(ii) an antifactor Xa activity of up to 10 IU/mg;
(iii) a weight average molecular weight from 6.5 to 9.5 kDa;
(iv) wherein the polysaccharide chains in said chemically modified heparin contain a predominant structure of the formula:

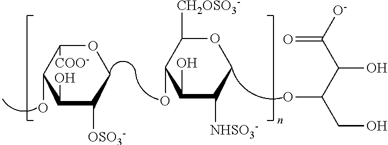

wherein n is an integer of from 2 to 25, such that it comprises from 2 to 25 disaccharide units corresponding to molecular weights from 1.2 to 15 kDa;
(v) retain at least 90% of the sulfate groups compared to native heparin;
(vi) have a reduction in chemically intact pentasaccharide sequences, responsible for providing an antithrombin mediated anticoagulant effect, compared to the polysaccharide chains of native heparin; and
(vii) have a reduction in unsulfated iduronic acid units and glucuronic acid units compared to native heparin.

Sevuparin may have in a 1H-NMR spectrum, no unidentified signals in the ranges 0.10-2.00 ppm, 2.10-3.10 ppm and 5.70-8.00 ppm larger than 4 percent when compared to the height of the signal present in native heparin at 5.42 ppm.

The predominantly occurring polysaccharide chains in sevuparin, have from 6 to 16 disaccharide units with molecular weights from 3.6 to 9.6 kDa, and may comprise glycol-split residues of the chemical structure:

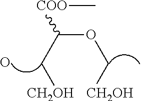

At least 30% of the polysaccharide chains of the compound sevuparin have a molecular weight of at least 8 kDa, 3-15% of the polysaccharide chains have a molecular weight of at least 15 kDa, 25-47% of the polysaccharide chains have a molecular weight of at least 9 kDa, 40-60% of the polysaccharide chains have a molecular weight of at least 7 kDa, 60-80% of the polysaccharide chains have a molecular weight of at least 5 kDa, at least 85% of the polysaccharide chains have a molecular weight of at least 3 kDa, and at least 95% of the polysaccharide chains have a molecular weight of at least 2 kDa.

The mean molecular weight of the major disaccharide is about 600 Daltons.

The manufacture of sevuparin is described in Examples 1 to 3 of the published patent application WO 2013/095276-A1.

The wording “endotoxemia” is defined as a condition characterized by the presence of endotoxins in the blood. On lysis, the outer cell wall of gram-negative bacteria enters the systemic circulation and initiates a pathophysiologic cascade of pro-inflammatory mediators. Serum endotoxin levels in a subject with endotoxemia are usually above a given threshold value, with detectable endotoxin levels of at least 5 pg/ml, generally at least 5-100 pg/ml, and more typically, endotoxin concentrations above 15-20 pg/ml. Endotoxemia raises many inflammatory cytokines in the body, such as Interleukin-1 (IL-1), Interleukin-6 (IL-6) and tumor necrosis factor-alpha (TNF-α). Endotoxemia comprises an increased level of LPS in the bloodstream at two to three times higher than normal concentrations. The level of endotoxins and inflammatory cytokines may differ slightly depending on the method used to measure these, however it will be clear to a person skilled in the art whether an increase in endotoxins as well as of any other inflammatory biomarker falls within the definition of endotoxemia regardless of the method used to measure said endotoxins and biomarkers.

The wording “endotoxin(s)”, is/are pyrogens that Gram-negative bacteria produce. In the field of bacteriology, this complicated compound is also referred to as lipopolysaccharide (LPS). It can be located in the membranes on the outside of bacteria such as Escherichia coli, Salmonella shigella, Vibrio cholerae as well as Haemophilus influenzae. Endotoxin is present in some, but not all patients with sepsis, as well as in many patients with acute life-threatening illnesses that would not meet the criteria for sepsis. A majority of patients meeting clinical criteria for sepsis can be shown to have elevated levels of circulating endotoxin; however, these elevated levels are also seen in a large number of acutely ill patients who do not meet sepsis criteria.

Early therapeutic intervention in patients documented to have endotoxemia or diagnosed as being at risk of developing endotoxemia is critical in order to prevent progression of endotoxemia into sepsis, septic shock, SIRS, MODS, ARDS, ALI, and mortality.

The wording “sepsis” is a life-threatening syndrome usually caused by bacterial infection, and refers to a systemic response to a culture-documented infection consisting of two of the following four criteria: temperature >38° C. or <36° C.; heart rate >90 beats per minute; respiratory rate >20 breaths/minute; white blood cell count >12 000 cells/mm. Sepsis involves an immune response that results in organ dysfunction or failure and is characterised by a respiratory rate of ≥22 breaths/minute, systolic blood pressure of ≤100 mm Hg and an altered level of consciousness. Severe sepsis is defined as sepsis complicated by organ dysfunction.

The wording “septic shock” is a subset of sepsis and is defined as sepsis-induced hypotension, persisting despite adequate fluid resuscitation, along with the presence of hypoperfusion abnormalities or organ dysfunction.

The wording “treatment or therapy” as used herein takes the normal wording within the medical and pharmaceutical field, and includes therapeutic treatment as well as prophylactic treatment (preventive treatment).

The wording “therapeutic treatment” as used herein means treatment of a subject with endotoxemia or an endotoxemia-associated condition such as SIRS, MODS, ARDS, and ALI, as herein described and claimed. This treatment (therapy) may be monotherapy with sevuparin alone, or combination therapy with sevuparin and a standard of care (SOC) agent as herein described.

The wording “prophylactic treatment (preventive treatment)” or “prophylactic therapy (preventive therapy)” as used herein means that sevuparin is used in a subject at risk of developing endotoxemia or an endotoxemia-associated condition such as SIRS, MODS, ARDS, and ALI. This treatment (therapy) may be monotherapy with sevuparin alone, or combination therapy with sevuparin and a standard of care (SOC) agent as herein described.

The wording “monotherapy” as used herein, means therapy (treatment) of endotoxemia as herein disclosed and claimed, with the compound sevuparin alone. Monotherapy may be therapeutic therapy (treatment) or prophylactic treatment (preventive) therapy (treatment).

The wording “combination therapy” as used herein means therapy (treatment) of endotoxemia as herein disclosed and claimed, wherein the compound sevuparin and a standard of care (SOC) agent are used in combination. Such combination therapy may be add-on therapy; combination therapy with co-administration of the compound sevuparin and a standard of care (SOC) agent; combination therapy where the compound sevuparin and a standard of care (SOC) agent are administered simultaneously; combination therapy where the compound sevuparin and a standard of care (SOC) agent are administered separately but in combination; or combination therapy where the compound sevuparin and a standard of care (SOC) agent are administered sequentially.

The wording “standard of care (SOC) agent” as used herein means a standard of care (SOC) agent used in the treatment of endotoxemia. Examples of SOC agents are: anti-coagulant agents (such as a heparin including but not limited to enoxaparin); an antibiotic; an additional endotoxin neutralizing agents such as a TLR-4 receptor antagonist; a cytokine inhibitor such as IL-6, an IL-1, or a TNF; an anti-inflammatory agent such as a non-steroidal agent (NSAID) including but not limited to salicylic acid or a COX-2 inhibitor; an anti-inflammatory agent such as a steroid including but not limited to prednisone, prednisolone, methylprednisolone, triamcinolone or dexamethasone. Standard of care (SOC) therapy for endotoxemia may also be a combination of one or more of the SOC agents listed herein.

The wording “add-on therapy” as used herein, is defined as combination therapy wherein the compound sevuparin is administered to a subject prior to administering a standard of care (SOC) agent, or wherein sevuparin is administered to a subject who is already being treated with a standard of care (SOC) agent.

The wording “simultaneous administration” as used herein, means that sevuparin and a standard of care (SOC) agent, are administered simultaneously.

The wording “co-administration” as used herein, means that sevuparin and a standard of care (SOC) agent, are administered at the same time, i.e. administered separately from each other but as combination therapy, to a subject in need of treatment or who is being treated.

The wording “sequentially” or “sequential administration” as used herein, means that the compound sevuparin is administered after the administration of a standard of care (SOC) agent, but could also mean that the compound sevuparin is administered prior to administration of a standard of care (SOC) agent.

The wording “pharmaceutical combination product” as used herein, means a drug combination product comprising the compound sevuparin and a standard of care (SOC) agent, where sevuparin and a standard of care (SOC) agent are provided separately but together as a “kit-of-parts combination product”.

The wording “a subject at risk of developing endotoxemia” as used herein means a subject with an ongoing disorder that has a heightened risk of developing endotoxemia as described and defined above.

The wording “gram-negative bacteria” refers to a group of bacteria that constitute one of the world's most significant public health problems due to their high resistance to antibiotics. These microorganisms have significant clinical importance in hospitals because they put patients in the intensive care unit (ICU) at high risk and lead to high morbidity and mortality. Two large groups, Enterobacteriaceae and the non-fermenters, are responsible for most clinical isolates; nevertheless, other clinically concerning gram-negative organisms exist, including but not limited to Neisseria, Haemophilus spp., Helicobacter pylori, and Chlamydia trachomatis. Enterobacteriaceae are a heterogeneous group widely dispersed in nature. They account for about 80% of gram-negative isolates with a myriad of disease-causing general/species in humans, including urinary tract infections, pneumonia, diarrhea, meningitis, sepsis, endotoxic shock, and many others. The general/species that frequently affect humans are Escherichia, Proteus, Enterobacter, Klebsiella, Citrobacter, Yersinia, Shigella, and Salmonella, among others. The non-fermenter, gram-negative bacilli (BNF) have a lower frequency of isolation when compared to Enterobacteriaceae; however, they are a relevant group since they cause severe, fatal infections, especially in the hospital environment. They also cause opportunistic diseases in ICU patients who undergo invasive procedures. The main BNF microorganisms that cause human disease are Pseudomonas aeruginosa, Acinetobacter baumannii, Burkholderia cepacia, Burkholderia pseudomallei, Stenotrophomonas., Alcaligenes, and Moraxella. Endotoxemia resulting from Gram-negative bacteria may also be associated with fever, systemic inflammation, disseminated intravascular coagulation, hypotension, acute renal failure, Acute respiratory distress syndrome, adult respiratory distress syndrome (ARDS), symptoms of hepatocyte destruction and/or cardiac dysfunction, various forms of septic shock (including but not limited to endotoxin shock).

The wording “gram-positive bacteria” are classified by the colour they turn after a chemical called Gram stain and which is applied to them. Gram-positive bacteria stain blue when this stain is applied to them, whereas gram-negative bacteria stain red. Gram-positive and gram-negative bacteria stain differently because their cell walls are different, and they also cause different type of infections. Examples of gram-positive bacteria belonging to Gram-positive bacilli are: Anthrax, Diphteria, Enterococcal infections, Erysipelothricosis, Listeriosis. Examples of gram-positive bacterial infections due to Gram-positive cocci are: Pneumococcal infections, Staphylococcal aureus infections such as by MRSA (methicillin-resistant Staphylococcus aureus), Streptococcal infections and Toxic shock syndrome.

The wording “a major surgical procedure” is defined as a procedure where a surgeon opens the body of a subject, allowing the surgeon access to the area where the work needs to be completed. It involves major trauma to the tissues, a high risk of infection, and an extended recovery period. Most major surgeries will leave a large scar. Examples of major surgical procedures are thoracic (cardiac) surgery, abdominal surgery and neurosurgical procedures.

The wording “a severe acute disease” means a disease or condition which occurs suddenly, and which is life threatening. Examples of such diseases are pancreatitis, cholecystitis, intestinal arterial obstruction, portal vein thrombosis, aortic aneurysm rupture, and aortic dissection.

The wording “hepatic failure”, commonly also referred to as liver failure, is the inability of the liver to perform its normal synthetic and metabolic functions as part of normal physiology. Two forms are recognised, acute and chronic (cirrhosis). Acute liver failure is defined as a rapid development of hepatocellular dysfunction, specifically coagulopathy and mental status changes (encephalopathy) in a patient without known prior liver disease. Chronic liver failure usually occurs in the context of cirrhosis, itself potentially the result of many possible causes, such as excessive alcohol intake, hepatitis B or C, autoimmune, hereditary and metabolic causes (such as iron or copper overload, steatohepatitis or non-alcoholic fatty liver disease).

The wording “cirrhotic subject” means a subject (patient) suffering from cirrhosis, also known as liver cirrhosis or hepatic cirrhosis, and end-stage liver disease. It is defined as a subject with an impaired liver function caused by the formation of scar tissue known as fibrosis due to damage caused by liver disease.

The wording “non-cirrhotic subject” means a subject (patient) with hepatic failure (liver failure) but where the subject (patient) has not yet developed cirrhosis.

The wording “hepatorenal syndrome” is a multiorgan condition affecting the kidneys and the liver. It is a cause of acute kidney injury that can be seen in those with acute or chronic liver disease.

The wording “intestinal endotoxemia (leaky gut)” or “leaky gut syndrome” means a condition wherein abnormal amounts of endotoxin are permitted to leak across the gastrointestinal mucosa into systemic circulation. This condition may exist as the result of many different types of systemic illness or injury.

The wording “metabolic endotoxemia” means presence of excess LPS (lipopolysaccharides) in the blood that produce no symptoms or moderate symptoms, thereby contributing to chronic metabolic disorders. High-fat and high-carbohydrate content (fast-food style western diet) increase intestinal permeability and LPS concentrations. The relationship between metabolic endotoxemia and the onset of diabetes, obesity, and heart disease is well established.

The wording “diet-induced endotoxemia” means endotoxemia which occurs due to a high-fat diet which in turn increases the level of LPS in a subjects (patients) blood.

The wording “portal endotoxemia” is a condition when endotoxins have entered into a subject's portal blood.

The wording “autoimmune disease” is a condition arising from an abnormal immune response to a functioning body part. Immune cells target the body's own healthy tissues by mistake, signaling the body to attack them. Examples of autoimmune diseases are inflammatory bowel disease (IBD), Crohn's Disease or ulcerative colitis.

The wording “systemic inflammatory response syndrome (SIRS)” is an exaggerated defense response of the body to a noxious stressor (infection, trauma, surgery, acute inflammation, ischemia or reperfusion, or malignancy, to name a few) to localize and then eliminate the endogenous or exogenous source of the insult. Objectively, SIRS is defined by the satisfaction of any two of the criteria below:
Body temperature over 38 or under 36 degrees Celsius;
Heart rate greater than 90 beats/minute;
Respiratory rate greater than 20 breaths/minute or partial pressure of CO2 less than 32 mmHg;
Leukocyte count greater than 12000 or less than 4000/microliters or over 10% immature forms or bands.

The wording “multiple organ dysfunction syndrome (MODS)” is the clinical consequence of a dysregulated inflammatory response, and is one of the most common syndromes of critical illness.

The wording “Acute Respiratory Distress Syndrome (ARDS)” is an acute, diffuse, inflammatory form of lung injury and life-threatening condition in seriously ill patients, characterized by poor oxygenation (hypoxia), pulmonary infiltrates, and acute onset. On a microscopic level, the disorder is associated with capillary endothelial injury and diffuse alveolar damage. ARDS is defined by the patient's oxygen in arterial blood (PaO₂) to the fraction of the oxygen in the inspired air (FiO₂). These patients have a PaO₂/FiO₂ ratio of less than 300 mm Hg. The definition of ARDS was updated in 2012 and is called the Berlin definition. ARDS is often divided into three categories of severity: mild ARDS with PaO₂/FIO₂≤300 mm Hg; moderate ARDS with PaO₂/FIO₂≤200 mm Hg; and severe ARDS with PaO₂/FIO₂≤100 mm Hg (The Berlin ARDS Definition Task Force; JAMA June 2012; 307 (23): pp. 2526-2533). It is also important to bear in mind that as clinicians learn more and more about endotoxemia-associated ARDS as time goes by, the experts in the field may develop the ARDS definition further and the wording ARDS as used in the present patent specification and claims, should be interpreted and defined accordingly.

“Acute Lung Injury (ALI)”, is the less severe form of acute respiratory failure, and is also defined according to the Berlin definition. ARDS as well as ALI may or may not involve sepsis or pneumonia, or a combination of both.

The wording “Endotoxemia-associated Acute Respiratory Distress Syndrome (ARDS)” or “Endotoxemia-associated Acute Lung Injury (ALI)”, as used throughout the present patent specification and claims, is ARDS or ALI as defined above and which is associated with endotoxemia. The more severe lung injury, the higher is the endotoxin concentration in a patient's blood serum.

Hypoxia is a condition in which the body or a region of the body such as the lung(s) is deprived of adequate oxygen supply. A patient having an O₂ saturation (SpO₂)<90% has hypoxia. The PaO₂/FiO₂ ratio is another way to measure the degree of hypoxia. A normal PaO₂/FiO₂ ratio is about 300 to 500 mmHg. The ratio of less than 300 indicates abnormal gas exchange, and values less than 200 mmHg indicate severe hypoxemia.

The wording tachypnoe (tachypnea), sometimes also called hyperventilation, is a respiratory rate greater than normal, resulting in abnormally rapid and shallow breathing (Whited L, Graham D D 2020 “Abnormal Respirations”; StatPearls Traesure Island (FL); StatPearls Publishing; PMID 29262235). In adult humans at rest, any respiratory rate of 12-20 per minute is considered clinically normal, with tachypnea being any rate above that (Dorland's Medical Dictionary). Children have significantly higher resting ventilatory rates, which decline rapidly during the first three years of life and then steadily until around 18 years. Tachypnoe (tachypnea) is often used as a predictor and/or indicator of hypoxia.

The wording “Covid-19” (Coronavirus disease) is an infectious disease caused by the SARS-COV-2 virus.

### Pharmaceutical Formulations and Administration Routes

The compound sevuparin as used in accordance with the present invention, may be administered as a pharmaceutical formulation. Suitable administration routes are systemically by parenteral administration, such as subcutaneous administration, intravenous injection or infusion.

A further aspect of the invention is oral administration of the compound sevuparin, when used in accordance with the present invention.

For parenteral administration, the compound sevuparin, may be incorporated into a solution or suspension, which may also contain one or more adjuvants such as sterile diluents such as water for injection, saline, fixed oils, polyethylene glycol, glycerol, propylene glycol or other synthetic solvents, antibacterial agents, antioxidants, chelating agents, buffers and agents for adjusting the osmolality. The parenteral preparation may be delivered in ampoules, vials, prefilled or disposable syringes also for self-administration, or as infusion arrangements, such as for intravenous or subcutaneous infusion.

Pharmaceutical compositions useful in therapy according to the invention, may comprise sevuparin, together with at least one conventional pharmaceutically and pharmacologically acceptable excipient and/or carrier. The excipient and/or carrier may be a solid, semisolid or liquid material that can serve as a vehicle for the active substance.

### EXAMPLES

### Manufacture of Sevuparin

Sevuparin, as used in accordance with the present invention, may be prepared by following the synthetic procedure as described in Examples 1 to 3 of the published patent application WO 2013/095276-A1.

### Clinical Phase 1 Trial

A randomized, placebo-controlled study to evaluate the effects of intravenous sevuparin on LPS reactions (first part), and the interaction between subcutaneous enoxaparin and sevuparin on coagulation responses in healthy volunteers (second part), has been performed.

LPS-induced lung injury is one of the most commonly used rodent models for ARDS (Matute-Bello et al., 2008: Animal models of acute lung injury. Am. J. Physiol. Lung Cell. Mol. Physiol. 295, L379-L399) and has been described to mimic the neutrophilic inflammatory response observed in ARDS patients (Matute-Bello et al., 2011: An official American thoracic society workshop report: features and measurements of experimental acute lung injury in animals. Am. J. Respir. Cell Mol. Biol. 44, 725-738).

LPS is a part of the outer membrane of Gram-negative bacteria and can be administered into the airways (direct, pulmonary insult) and systemically (extra-pulmonary insult) (Menezes et al., 2005: Pulmonary and extrapulmonary acute lung injury: inflammatory and ultrastructural analyses. J. Appl. Physiol. 98, 1777-1783).

The animal LPS model is thus a well-recognised model for Acute Respiratory Distress Syndrome (ARDS) associated with endotoxemia and for Acute Lung Injury (ALI) associated with endotoxemia.

Objectives for first part of the clinical study: To evaluate the effect of intarvenous (IV) sevuparin on safety and tolerability and inflammatory responses following an intravenous (IV) LPS challenge.

### Endpoints

### Microvascular Function

Laser speckle contrast imaging with heating protocol
Sidestream Darkfield Imaging.
Passive limb movement

### Blood

Leucocyte differential
Cytokines (including IL-6, IL-8, IL-10 and TNF-α)
Flow cytometry (Neutrophils, monocyte subsets, T cells, B cells, NK cells, dendritic cells, including neutrophil extracellular traps)
Explorative biomarkers: acute phase reactants and components measurable in blood, serum and plasma of relevance for septic inflammation may be measured and reported separately.

### Pharmacokinetics

### Safety and Tolerability

Vital signs
Treatment-Emergent Adverse Events
Electrocardiography
Haematology and chemistry blood panels, including HIT antibodies
NRS (self-rated sickness feeling)

Objectives for the second part of the clinical study: Comparison of subcutaneous (s.c.) enoxaparin and s.c. sevuparin on coagulation parameters; to evaluate the safety/tolerability of sevuparin in interaction with enoxaparin.

### Endpoints

### Coagulation Parameters Including APTT, PT, INR, Anti-fXa, Anti-Flla and D-Dimer

### Safety and Tolerability

Vital signs
Treatment-Emergent Adverse Events
Electrocardiography
Haematology and chemistry blood panels, including HIT antibodies

48 volunteers were randomized over 4 treatment groups in total in the clinical study:
sevuparin low dose (n=12)
sevuparin intermediate dose (n=12)
sevuparin high dose (n=12)
placebo (n=12)

In the second part of the study, two groups of eight subjects receives a single dose of enoxaparin and either a single dose of sevuparin or a single dose of placebo in a cross-over fashion with an intervening wash-out period of at least 8 days.

### Study Drug (Investigational Medical Product, IMP)

In the experiments performed below, sevuparin was provided by Modus Therapeutics AB, Sweden, as a 150 mg/ml solution formulated in a 15 mM phosphate buffer at a pH of 7.0. The non-preserved sterile solution is dispensed (5.4 mL overfill) in glass vials sealed with a rubber stopper and covered with a tear-off aluminium cap.

The placebo product is a solution for IV infusion or subcutaneous injection supplied in single-dose 10 mL type I glass vials containing 5.4 mL of 9 mg/ml sodium chloride (overfill) in a glass vial sealed with a rubber stopper and covered with a tear-off aluminium cap. The placebo product is identical in appearance to the active drug.

In the first part of the study, sevuparin (or matching placebo) is administered by IV infusion (bolus loading dose followed by continuous infusion over 6 hours); In the second part of the study, sevuparin and enoxaparin are given by subcutaneous injection.

The sevuparin dose was as follows in the first part of the study:
Group 1 (sevuparin low dose): loading bolus dose of 0.3 mg/kg bodyweight, followed by IV infusion at 0.08 mg/kg/h;
Group 2 (sevuparin intermediate dose): loading dose of 1 mg/kg bodyweight, followed by IV infusion at 0.25 mg/kg/h;
Group 3 (sevuparin high dose): loading dose of 3 mg/kg bodyweight, followed by IV infusion at 0.75 mg/kg/h.

In the second part of the study, the dose of enoxaparin is 40 mg. The dose of sevuparin is 6 mg/kg. Both enoxaparin and sevuparin/placebo are administered subcutaneously.

### Interaction Drug/Challenge Agent (NIMP-Non-Investigational Medical Product)

Enoxaparin 40 mg (400 IU) in single subcutaneous doses.

LPS, purified lipopolysaccharide prepared from Escherichia Coli: 113: H10: K negative (U.S. Standard Reference Endotoxin) is used. This LPS batch is manufactured in the US by List Biological Laboratories.

In the first part of the study, 1 ng/kg bodyweight of LPS was administered intravenously.

### Subjects/Groups

In the first part of the study, a total of 48 male and female healthy volunteers were enrolled and randomized over 4 treatment groups (sevuparin low, intermediate or high dose or matching placebo). Each treatment group consisted of 12 healthy volunteers. All subjects underwent an intravenous LPS challenge.

In the second part of the study, 16 subjects receive two single doses of enoxaparin, and two single doses of sevuparin or placebo.

### Inclusion Criteria

1. Healthy male and female volunteers aged 18 to 55 years, inclusive. Health status is defined by absence of evidence of any active or chronic disease following a detailed medical and surgical history, a complete physical examination including vital signs, 12-lead ECG, haematology, blood chemistry, and urinalysis;
2. BMI in the range of 18 to 30 kg/m², a minimum body weight of 50 kg and a maximum body weight of 112 kg;
3. Be able to abstain from smoking from 24 hours prior to dosing until study discharge visit;
4. No history of alcohol or drug abuse;
5. No history of trauma with likely damage to the spleen or surgery to spleen; 6. Free from any clinically significant febrile illness 30 days preceding study Day 1; 7. Non-atopic constitution, including non-asthmatic;
8. No use of any prescription drugs, including aspirin or other non-steroid anti-inflammatory drugs;
9. Able to give written informed consent and willing to comply with all study-related procedures;
10. Female subjects of childbearing potential and male subjects who have sexual intercourse with a woman of childbearing potential must be willing to practice effective contraception during the study and be willing and able to continue contraception for at least 90 days after their last dose of study treatment. Women of childbearing potential are defined as all women physiologically capable of becoming pregnant, unless they meet one of the following conditions:
   Postmenopausal: 12 months of natural (spontaneous) amenorrhea or 6 weeks after surgical bilateral oophorectomy with or without hysterectomy;
   Posthysterectomy.

For the purposes of the study, effective contraception is defined as follows:
Females: Using 1 or more of the following acceptable methods of contraception: surgical sterilization (e.g., bilateral tubal ligation), intrauterine contraception/device, hormonal contraception, or any 2 barrier methods (a combination of male or female condom with diaphragm, sponge or cervical cap).
Males: Effective male contraception includes a vasectomy with negative semen analysis at follow up, or the use of condoms.

Abstinence can be considered an acceptable method of contraception at the discretion of the investigator. Periodic abstinence (e.g., calendar, ovulation, symptothermal, post ovulation methods) and withdrawal are not considered acceptable methods of contraception.

### Exclusion Criteria

1. History of sepsis or history of clinically significant cardiovascular disease, syncope or malignancy;
2. Reported unintended weight loss or gain of at least 5 kg in four weeks at screening;
3. Haemorrhagic diathesis (easy bruising, epistaxis, gastro-intestinal bleeding);
4. Clinical evidence of significant or unstable medical illness including neurological, hematological (including von Willebrand disease and heparin-induced thrombocytopenia, HIT), cardiovascular (including clinically significant arrhythmia), hepatic, pulmonary, metabolic, gastrointestinal, renal, psychiatric, endocrine or infectious diseases or malignancies. Subjects who have had splenectomy.
5. First degree family history of premature cardiovascular disease event (if diagnosed before 50 years of age);
6. Previous participation in a systemic (i.v./inhaled) LPS challenge trial or prior exposure to systemic endotoxin within a year before the first study day (applicable to Part 1 and 2 only) or previous exposure to sevuparin in study Part 1 or 2 (applicable to Part 3 only);
7. Antibiotic use, operation or intervention by surgeon/dentist within one month before the first study day;
8. Subjects who have received any prescribed systemic or topical medication within 14 days of the first dose administration unless in the opinion of the Investigator the medication will not interfere with the study procedures or compromise safety;
9. Subjects who have received any of the following excluded medications within prescribed 14 days of the first dose administration: aspirin, anti-platelet therapy, anticoagulant therapy and prophylactic and therapeutic LMWH or un-fractioned heparin;
10. Subjects who have used any non-prescribed systemic or topical medication (including herbal remedies) within 7 days of the first dose administration (with the exception of vitamin/mineral supplements) unless, in the opinion of the Investigator, the medication will not interfere with the study procedures or compromise safety.
11. Subjects who have received any medications, including St John's Wort, known to chronically alter drug absorption or elimination processes within 30 days of the first dose administration unless, in the opinion of the Investigator, the medication will not interfere with the study procedures or compromise safety;
12. Any active inflammatory or infectious disease (e.g. periodontitis);
13. Hypertension (defined as systolic blood pressure >140 mmHg or diastolic blood pressure >90 mmHg, repeatedly measured after 5 minutes in resting supine position);
14. Hypotension (defined as systolic blood pressure <90 mmHg or diastolic blood pressure <50 mmHg);
15. Clinically significant abnormalities on the 12-lead ECG (QRS complex>120 ms, PR interval >210 ms, QTcF interval >470 ms);
16. Screening transaminases (AST, ALT, GGT) ≥1.5 times the ULN; estimated glomerular filtration rate (GFR, MDRD equation)<60 mL/min; APTT above the normal range, INR above 1.4; absolute platelet count <150,000/μL.
17. Positive test results for Hepatitis B, Hepatitis C, HIV antibody or any other obvious disease associated with immune deficiency;
18. Subjects who have received prophylactic/therapeutic LMWH or un-fractioned heparin within the last year;
19. Biochemical diagnosis of diabetes mellitus;
20. Biochemical diagnosis of hypo- or hyperthyroidism (TSH <0.3 or >4.8 mU/L);
21. Subjects who consume more than 3 units of alcohol per day (one alcohol unit=1 beer [12 oz]=1 wine [5 oz]=1 spirits [1.5 oz]);
22. Subjects with a positive urine drug screen/alcohol test result at screening or first admission or a history of substance abuse in the last 12 months prior to the start of the study;
23. Subjects who smoke more than 6 cigarettes or the equivalent in tobacco per day and are unwilling to abstain from smoking from 24 hours prior to dosing until study discharge visit.
24. Other medical or psychological conditions which, in the opinion of the investigator, might create undue risk to the subject or interfere with the subject's ability to comply with the protocol;
25. Donation of blood within 3 months prior to screening or donation of plasma within 14 days prior to screening;
26. Participation in an investigational drug or device study within 3 months between last dosing in previous study and first dosing in present study or more than 4 times in the past year;
27. Not having a general practitioner;
28. Not willing to accept information transfer which concerns participation in the study, or information regarding health, like laboratory results, findings at anamnesis or physical examination and eventual adverse events to and from his general practitioner;
29. Not willing to give permission to have the general practitioner to be notified upon participation in this study;
30. Any vaccination within the last 3 months; COVID19 vaccination (or time of infection) is allowed up until 4 weeks prior to the first sevuparin/placebo dosing;
31. Subjects who have any current and/or recurrent pathologically, clinically significant skin condition at the lower forearms (i.e. atopic dermatitis); including tattoos (applicable to Part 1 and 2 only);
32. Subject is female and is pregnant (based upon serum pregnancy test at screening and urine pregnancy test pre-dose to the first sevuparin/placebo administration), breast-feeding, or planning to become pregnant during the study or within 90 days after last dose of study treatment.

### Treatment Duration

The total study duration for each subject was 64 days, divided as follows:

### First Part of Study

IV LPS challenge: Day-1 to 3 (admission Day-1 to 2)
EOS: Day 8 (up to Day 10)

There was a washout period of at least 8 days between study drug administration.

### Second Part of Study

Screening: Up to 42 days before dosing
Enoxaparinsevuparin interaction
   Period 1: Day-1 to 3 (admission Day-1 to 2)
   Period 2: Day-1 to 3 (admission Day-1 to 2)
   A washout period of at least 8 days between study drug administrations in period 1 and 2.
EOS: Day 8 (up to Day 10).

In both parts of this clinical study, subjects are discharged approximately 24-30 hours after end of study drug administration.

### Primary Endpoint

The primary goal of the first part of this study was to investigate the safety and tolerability of sevuparin during induced systemic inflammation (IV LPS).

The primary endpoint for the first part of the study (IV LPS) is safety and tolerability, while changes in immune cells and inflammatory cytokines are key secondary endpoints in this part.

In addition, based on these endpoints, a dose-response regimen is evaluated in order to determine the optimal sevuparin dose for subsequent clinical trials and ultimately for future clinical use.

The primary goal of the second part of the study is to assess magnitude of any pharmacodynamic interactions in terms of coagulation parameters when sevuparin or placebo is given together with enoxaparin.

### Screening

The Screening Visit may be conducted anytime up to 42 days prior to first study drug administration. The screening phase will only be started after full written, verbal and signed informed consent has been obtained, according to CHDR standard operating procedures. A full medical screening will be performed to assess a subject's eligibility for this study.

### Re-Screening

It is permitted to re-screen subjects, if the reason for non-eligibility is considered transient (e.g., abnormal laboratory test, insufficient washout period of a forbidden medication, positive drug screen, etc.). During a re-screening, only assessments that are susceptible to change within the timeframe between screenings are repeated (e.g. medical history, demographics, virology are not repeated).

### Treatment and Observation Period

Subjects will present to the CRU for baseline procedures conducted prior to dosing on Day 1. During the second part of the study, subjects undergo intravenous sevuparin/placebo and LPS treatment on Day 1. In the morning of Day 2, 24-30 hours after start, sevuparin/placebo treatment, subjects returns home. At 48 hours after start sevuparin treatment, subjects returns to CHDR for outpatient assessments.

During the second part of the study, in the morning of Day 1, subjects will receive subcutaneous injections of enoxaparin and sevuparin or placebo. In the morning of Day 2, 24-30 hours after sevuparin/placebo treatment, subjects will return home. At 48 hours after sevuparin treatment, subjects will return to CHDR for outpatient assessments.

In general, subjects will begin overnight fasting period at least 10 hours prior to sevuparin/placebo dosing. Water will be allowed ad libitum.

### Follow-Up/EOS

In both the first and second part of the study, subjects will return for End of Study (EOS) visit at 8-10 days after last study drug administration. EOS is defined as the last visit of the last subject. Upon early termination, the EOS visit will also be performed approximately 8-10 days after the last study drug administration. During this visit, assessment of platelets count and clinical HIT score will be calculated. In the event of IMP-induced HIT, samples will be analysed for HIT-induced antibodies and in the case that subjects are found positive, they will be followed-up for clinical and HIT assessments at 3- and 6-months timepoints.

### Concomitant Medications

No prescription medications, OTC medications, vitamin, herbal and dietary supplements will be permitted within 7 days prior to study drug administrations, or less than 5 half-lives (whichever is longer), and during the course of the study. Exception is paracetamol (up to 4 g/day). Allowance of use of other medications will be determined upon discretion of the investigator.

### Lifestyle Restrictions

Approximate mealtimes will be according to the study schedule. In-clinic meals will be comparable in composition and time of administration across all groups in each study part.
For each visit (except Part 1 FU visit), subjects will be required to fast for at least 4 hours. Subjects will be required to fast minimally 10 hours overnight (no food and no fluid) before study dose administration. Water is allowed as required.
The use of (illicit) drugs including cannabis can influence the measurements. Therefore, using ‘drugs’ is not permitted from 3 days before dosing and until EOS. Since poppy seeds can cause a positive ‘drugs’ result; these should be avoided. If a positive result occurs without an explanation, the subject cannot participate in the study. However, positive urine drug screen for prescribed medication is allowed at the discretion of the PI.
Alcohol will not be allowed from at least 24 hours before each visit, and whilst in the study unit. At other times throughout the study, subjects should not consume more than 2 units of alcohol daily on average (one unit is 10 grams of alcohol). Subjects may undergo an alcohol breath test at the discretion of the investigator.
Subjects will not be allowed to have excessive caffeine consumption, defined as >800 mg per day from 7 days prior to the first dose of the study drug until 24 hours prior to dosing. Subjects will abstain from caffeine-containing products for 24 hours prior each visit until discharge from the study unit. Caffeine quantities defined as: one cup of coffee contains 100 mg of caffeine; one cup of tea, or one glass of cola, or portion of chocolate (dark: 100 g, milk 200 g) contains approximately 40 mg of caffeine; one bottle of Red Bull contains approximately 80 mg of caffeine.
Subjects will abstain from the use of tobacco- or nicotine-containing products (including e-cigarettes and patches) for 24 hours prior to dosing until EOS.
Strenuous physical activity (e.g., heavy lifting, weight or fitness training) is not allowed from 48 hours prior to each study day until EOS. Light ambulatory activities (e.g. walking at normal pace) will be permitted, with the level of activities kept as similar as possible on all days in the study unit.

### Interaction Drug/Challenge Agent (NIMP)

### Enoxaparin

Low molecular weight heparins (LMWHs) are widely used as thrombosis prophylaxis in bed-ridden patients in medicine clinics, including sepsis patients. Although there are a number of different entities in use globally, enoxaparin is chosen here since it is one of the most frequently used worldwide. In the present study (second part), enoxaparin was supplied by Techdow Pharma Netherlands B.V.

### Intravenous LPS

In the first part of the current study, subjects are administered with intravenous LPS 30 min after the start of the infusion of sevuparin on study Day 1. LPS, purified lipopolysaccharide prepared from Escherichia Coli: 113: H10: K negative (U.S. Standard Reference Endotoxin) is used. This LPS batch was manufactured in the US by List Biological Laboratories. All participants received 1 ng/kg E. coli purified LPS, administered as a 2-minute infusion. To ensure that participants stay adequately hydrated, additionally saline (NaCl 0.9%) or glucose/saline was infused (2.5% glucose/0.45% sodium chloride) starting approximately 2 hours before LPS administration at a rate of 750 ml/h, until 6 h afterwards at a rate of 150 ml/h (Hijma et al., 2020). During infusion of study drug or placebo, infused over 6 hours at an infusion rate of 80 mL/h, the infusion rate of glucose/saline was lowered to 80 mL/h.

### Safety and Tolerability Assessments

### Vital Signs

Evaluations of systolic and diastolic blood pressure, pulse rate, respiratory rate, and temperature is performed throughout the study. Pulse and blood pressure is taken after 5 minutes in the supine position. Automated oscillometric blood pressures and pulse rate is measured using a Dash 3000,

### Dash 4000, Dynamap 400 or Dynamap ProCare 400.

### Weight and Height

Weight (kg) is recorded at screening and the follow-up visit or upon early termination. Height (cm) is recorded and body mass index (BMI) calculated at screening.

### Physical Examination

Physical examination (i.e., inspection, percussion, palpation and auscultation) is performed during the course of the study. Clinically relevant findings that are present prior to study drug initiation is recorded with the subject's Medical History. Clinically relevant findings found after study drug initiation and meeting the definition of an AE (new AE or worsening of previously existing condition) is recorded.

### Electrocardiography

ECGs are obtained during the course of the study using Marquette 2000/5500 and stored using the MUSE Cardiology Information System. ECGs are taken after at least 5 minutes in the supine position. When timings coincide, ECGs are performed before blood sampling. The investigator assesses the ECG recording as ‘normal’, ‘abnormal—not clinically significant’, or ‘abnormal—clinically significant’ and include a description of the abnormality as required. The ECG parameters assessed includes heart rate, PR, QRS, QT, and QTcF (calculated using Fredericia's method).

### NRS

Subjective assessment of feeling sick is recorded by the subjects during the first part of the study (IV LPS infusion) on a numeric rating scale (NRS) electronically.

### Laboratory Assessments

Blood and other biological samples are collected for the following clinical laboratory tests: Haematology; Chemistry and electrolytes; Glucose; Coagulation (after 4-hours fasting); Coagulation (conjugated bilirubin is reported only when total bilirubin is outside the reference range); Virology; Urinalysis; Pregnancy (for women, serum pregnancy test is performed at screening and urine pregnancy test at other visits when scheduled, and if pregnancy is suspected during the study); Alcohol screening; and Urine drug screening.

### Assessment of HIT Response

Samples for analyses for HIT antibodies are taken. Additional samples may be taken at 3 and 6 months if required to follow up any positive results. The clinical assessment on the probability of HIT is based on blinded data and the “4T assessment”, see table below.

In assessing suspected reactions, a score of >6 suggests a high probability of HIT; 4-5 points suggest an intermediate probability, while score ≤3 points imply a low probability of HIT. Other causes for thrombocytopenia may include LPS administration (CHDR data on file).

### Pharmacokinetic and Pharmacodynamic Assessments

### PK Samples Sevuparin

Approximately 3.5 mL blood is collected via an i.v. catheter placed in an antecubital vein in the arm (not used for infusion of study drug) in CTAD Vacutainer tubes. The exact actual clock time of withdrawal of the blood sample is recorded.

### Cytokines

Blood is collected at time points as shown in Table 1 below for the assessment of cytokines. Venous blood (4 mL) is collected in K2EDTA tubes. Plasma is collected after centrifugation at 2000×g for 20 minutes at room temperature and stored at −80° C., in 3×0.5 ml aliquots until analysis. Samples are analysed by Ardena Laboratories Assen.

TABLE 1

| DAY | TIME POINT |
|---|---|
| −1 | −17 h |
| 1 | −1.5 h; 0 h; 0.5 h; 1 h; 1.5 h; 2 h; 2.5 h; 3.5 h; 4 h; 4.5 h; |
| | 5.5 h; 6 h; 6.5 h; 7 h; 8 h; 8.5 h; 9.5 h; 12 h |
| 2 | 24 h |
| 3 | 48 h |
| EOS | +8-10 days |

### Coagulation Parameters (BsCoag2)

In the second part of the study, coagulation parameters including anti-fXa, anti-flla and D-dimer are assessed at time points as shown in Table 2 below. Venous blood is collected in 3.5 mL CTAD collection tubes and mixed by gently inverting the tubes 10 times and then placed in an ice/water bath. The samples are centrifuged, within 1 hour of collection, at 1500 g for 10 minutes at 4° C. The separated plasma is transferred into a suitably labelled polypropylene tube, and then centrifuged again at 1500 g for 10 minutes at 4° C. The plasma in the tube is aspirated (without disturbing the pellet at the bottom of the tube) and transferred into 3 labelled tubes for storage, within 2 hours of collection, at −20° C., pending shipping to the analytical laboratory.

TABLE 2

| DAY | TIME POINT |
|---|---|
| Screening | Up to −42 days; Day-1 |
| 1 | Pre-dose; 0 h; 15 min; 1 h; 2 h; 3 h; 4 h 6 h 8 h; 9 h; 12 h |
| 2 | 24 h |
| 3 | 48 h |
| EOS | +8-10 days |

Results from the Clinical Study

Throughout the Figures and tables related to the clinical study test results, the following abbreviations and its definitions are used:
IV LPS Placebo means subjects who have been challenged with intravenous LPS and thereafter received placebo intravenously as described above.
Sevuparin 0.3/0.08 means (sevuparin low dose): loading bolus dose of 0.3 mg/kg bodyweight, followed by 0.08 mg/kg/h intravenously;
Sevuparin 1/0.25 means (sevuparin intermediate dose): loading dose of 1 mg/kg bodyweight, followed by 0.25 mg/kg/h intravenously;
Sevuparin 3/0.75 means (sevuparin high dose): loading dose of 3 mg/kg bodyweight, followed by 0.75 mg/kg/h intravenously.

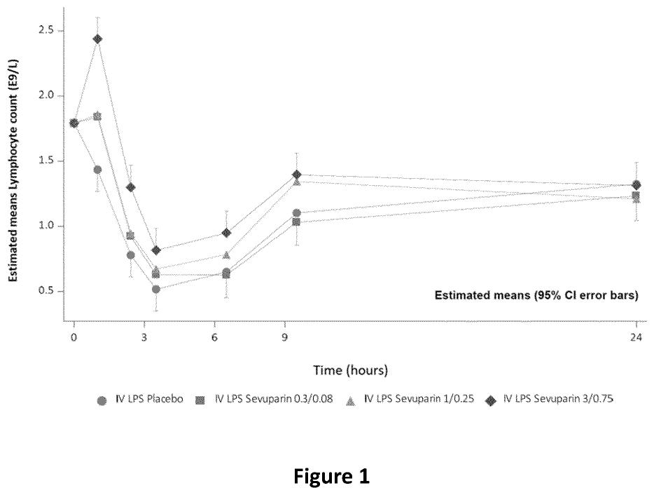

FIG. 1 is a graph showing the lymphocyte count from repeated blood samplings over time in subjects having received LPS intravenously, and simultaneously treatment by a continuous intravenous infusion of either placebo or three different doses of intradermal sevuparin.

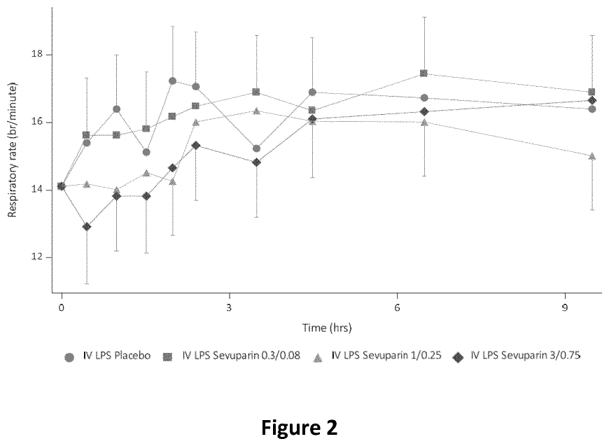

FIG. 2 is a graph showing the respiratory rate over time in subjects having received either of placebo or one of three different doses of sevuparin intravenously (first part of the study). As shown in the graph of FIG. 2, at the highest intravenously (IV) administered dose of sevuparin, there was an improvement of the respiratory rate compared to placebo.

Endotoxemia (LPS) consistently causes a rapid increase in respiratory rate (even to tachypnoe >20/min) of a subject and in the clinical setting, tachypnoe is a symptom that signals increasing severity of a critical condition.

Moreover, sevuparin did not have any therapeutic impact on heart rate, systolic blood pressure, diastolic blood pressure, body temperature or feeling sick, which is shown in Table 3 below.

TABLE 3

| | LS Means | | | | |
|---|---|---|---|---|---|
| | | Sevuparin | Sevuparin | Sevuparin | |
| | Placebo | IV LPS | IV LPS | IV LPS | |
| Parameter | IV LPS | 0.3/0.08 | 1.0/0.25 | 3.0/0.75 | P-value |
| Heart rate | 67.4 | 69.7 | 71.1 | 70.5 | 0.3885 |
| [bpm] | | | | | |
| Systolic blood | 116.1 | 115.5 | 117.3 | 114.0 | 0.1284 |
| pressure | | | | | |
| [mm Hg] | | | | | |
| Diastolic blood | 63.3 | 63.5 | 63.9 | 62.7 | 0.8411 |
| pressure | | | | | |
| [mm Hg] | | | | | |
| Body temperature | 36.96 | 37.13 | 36.93 | 36.97 | 0.2551 |
| NRS feeling sick | 1.4 | 1.9 | 1.9 | 2.0 | 0.4046 |

Also, sevuparin had no therapeutic effect on the inflammatory biomarkers cytokines and C-Reactive Protein (CRP). However, sevuparin produced a dose dependent pronounced increase in some white blood cell types (neutrophils, lymphocytes and basophils) compared to placebo control which is indicative of a potentially important immunomodulatory effect in patients with endotoxemia or at risk of endotoxemia. These results are shown in FIG. 1 (lymphocytes) and in Table 4 below.

TABLE 4

| | LS Means | | | | |
|---|---|---|---|---|---|
| | | Sevuparin | Sevuparin | Sevuparin | |
| | Placebo | IV LPS | IV LPS | IV LPS | |
| Parameter | IV LPS | 0.3/0.08 | 1.0/0.25 | 3.0/0.75 | P-value |
| IL-10 in plasma | 8.869 | 8.125 | 11.825 | 7.527 | 0.1924 |
| [pg/ml] | | | | | |
| IL-6 in plasma | 15.221 | 19.587 | 16.012 | 13.712 | 0.7108 |
| [pg/ml] | | | | | |
| IL-8 in plasma | 17.866 | 19.393 | 19.843 | 17.820 | 0.9597 |
| [pg/ml] | | | | | |
| TNF-α in plasma | 3.972 | 5.574 | 5.415 | 5.039 | 0.4369 |
| [pg/ml] | | | | | |
| C-Reactive | 9.406 | 10.893 | 11.080 | 11.853 | 0.4988 |
| Protein | | | | | |
| [mg/ml] | | | | | |
| Eosinophil count | 0.080 | 0.114 | 0.105 | 0.117 | 0.3692 |
| [E9/L] | | | | | |
| Basophil count | 0.031 | 0.029 | 0.032 | 0.039 | 0.0008 |
| [E9/L] | | | | | |
| Neutrophil count | 5.996 | 6.650 | 5.788 | 7.073 | 0.0117 |
| [E9/L] | | | | | |
| Lymphocyte | 0.968 | 1.046 | 1.136 | 1.369 | 0.0005 |
| count | | | | | |
| [E9/L] | | | | | |
| Monocyte count | 0.390 | 0.335 | 0.339 | 0.376 | 0.4431 |
| [E9/L] | | | | | |

1-28. (canceled)

29. A method for the treatment of endotoxemia, comprising administering a therapeutically effective amount of the compound sevuparin or a pharmaceutically acceptable salt thereof, to a subject in need of such treatment.

30-31. (canceled)

32. The method of claim 29, wherein the treatment is prophylactic treatment in a subject at risk of developing endotoxemia.

33. The method of claim 29, wherein the treatment is prophylactic treatment in a subject at risk of developing endotoxemia-associated Acute Respiratory Distress Syndrome (ARDS).

34. The method of claim 29, wherein the treatment is prophylactic treatment in a subject at risk of developing endotoxemia-associated Acute Lung Injury (ALI).

35. The method of claim 29, wherein the treatment is in a subject suffering from endotoxemia-associated Acute Respiratory Distress Syndrome (ARDS).

36. The method of claim 29, wherein the treatment is in a subject suffering from endotoxemia-associated Acute Lung Injury (ALI).

37. The method of claim 29, wherein the endotoxemia occurs in conjunction with infection by gram-negative bacteria or gram-positive bacteria.

38. The method of claim 29, wherein the endotoxemia occurs in conjunction with sepsis or septic shock.

39. The method of claim 29, wherein the endotoxemia occurs in conjunction with a major surgical procedure.

40. The method of claim 39, wherein the major surgical procedure is a thoracic (cardiac) surgery, an abdominal surgery or a neurosurgical procedure.

41. The method of claim 29, wherein the endotoxemia occurs in conjunction with a severe acute disease.

42. The method of claim 41, wherein the severe acute disease is pancreatitis, cholecystitis, intestinal arterial obstruction, portal vein thrombosis, aortic aneurysm rupture, or aortic dissection.

43. The method of claim 29, wherein the endotoxemia occurs in conjunction with an injury caused by a major trauma.

44. The method of claim 29, wherein the endotoxemia occurs in conjunction with hepatic failure.

45. The method of claim 44, wherein the hepatic failure is present in a non-cirrhotic subject.

46. The method of claim 44, wherein the hepatic failure is present in a cirrhotic subject.

47. The method of claim 44, wherein the endotoxemia occurs in conjunction with hepatorenal syndrome.

48. The method of claim 29, wherein the endotoxemia occurs in conjunction with intestinal endotoxemia (leaky gut).

49. The method of claim 29, wherein the endotoxemia is portal endotoxemia.

50. The method of claim 29, wherein the endotoxemia is present in a subject having an autoimmune disease.

51. The method of claim 50, wherein the autoimmune disease is inflammatory bowel disease (IBD), Crohn's Disease or ulcerative colitis.

52. The method of claim 29, wherein the endotoxemia is present in a subject having systemic inflammatory response syndrome (SIRS), Multiple organ dysfunction syndrome (MODS), Covid-19, or pneumonia.

53. The method of claim 29, wherein said use is combination treatment with standard of care (SOC) therapy for endotoxemia.

54. The method of claim 56, wherein the standard of care (SOC) therapy for endotoxemia is a low molecular weight heparin (LMWH).
